(19) United States
(12) Patent Application Publication
(10) Pub. No.: US 2008/0274519 A1
(43) Pub. Date: Nov. 6, 2008

(54) FLAVONOID CONCENTRATES

(75) Inventor: Robert Gerard Wallace, Willetton (AU)

Correspondence Address: MERCHANT & GOULD PC, P.O. BOX 2903, MINNEAPOLIS, MN, 55402-0903, US

(73) Assignee: BIORES HEALTH LIMITED, West Perth (AU)

(21) Appl. No.: 12/168,591

(22) Filed: Jul. 7, 2008

Related U.S. Application Data
(63) Continuation of application No. 10/482,637, filed on Jun. 14, 2004, now abandoned.

(30) Foreign Application Priority Data
Jun. 29, 2001 (AU) PR 6022

(51) Int. Cl. C12P 17/06

(52) U.S. Cl. 435/125

(57) ABSTRACT

A method of producing a flavonoid aglycone concentrate from plant material containing a suitable flavonoid glycoside and/or conjugate thereof comprising the steps of:
(i) enzymatically converting the flavonoid glycoside or conjugate thereof into the flavonoid aglycone; and
(ii) adjusting the pH to render the flavonoid aglycone relatively insoluble and forming a concentrate containing the same.

### FIELD OF THE INVENTION

The present invention relates to a method of preparing flavonoid aglycone concentrates from starting material containing a flavonoid glycoside and/or conjugate thereof. More particularly, the present invention provides an efficient method of producing enriched flavonoid aglycones concentrates from plant material using aqueous solvents.

### BACKGROUND ART

Flavonoids are a class of phytochemicals with wide ranging applications including their use as therapeutics, anti-microbials and antioxidants. They are capable of treating and or preventing a range of medical disorders and diseases including degenerative diseases such as heart disease, Alzheimer's disease, dementia and cancer, to mention a few. The characteristics and properties of flavonoids are well documented in the scientific literature.

The demand for ‘natural’ phytochemical remedies is increasing and will increase further as the average age of the world population steadily increases. Furthermore, the younger sections of the population are turning more to natural alternatives for treating or preventing medical conditions. In addition, there is a strong demand for such materials to be free of organic solvent residues, particularly those that are industrially synthesised, and for products produced with minimum burden to the environment. Society is also placing a high value on the use of biodegradable materials and processes that have minimal environmental impact.

The flavonoids are a sub-group of the plant polyphenols, double or triple ringed structures consisting of a basic fifteen carbon atoms skeleton. Plant flavonoid aglycones (i.e. flavonoids without attached sugars) occur in a variety of structural forms. However, all contain fifteen carbon atoms in their basic nucleus and these are arranged in a C₆-C₃-C₆ configuration, that is two aromatic rings linked by a three carbon unit which may or may not form a third ring.

The important role of flavonoids in diet and medicine is becoming more and more recognised. It is the flavonoids in red wine, green tea, extra virgin olive oil, soy products, fruit and vegetables, various traditional herbal medicines teas and tinctures that are at least partly responsible for the benefits gained from their consumption.

One group of flavonoids whose value is well established is the isoflavones. The isoflavones have a characteristic structure and form a particular isomeric class of flavonoids. The interest in isoflavones has been extensive including the suggestion that they are the factor in traditional oriental diets responsible for the lower incidence of breast and prostrate cancers in some populations of the eastern Asian region.

The isoflavones while appearing in other plant families are most strongly associated with the legumes, in particular with the Papilionoideae subfamily of the Leguminosase which includes many well known fodder crops such as clover, pulses—beans, soy beans, and peas, and shrubs such as gorse and broom.

In addition to the benefits of isoflavones to human and animal health, there has recently been shown application in the animal feeds industry where swine administered feed supplemented with isoflavones showed increased average daily weight gains, but no increase in feed intake. The pigs also had increased percentages of carcass muscle and higher estimated muscle gain per day.

While in an ideal world we would all obtain enough of these compounds from the careful selection of foods, meals and drinks, in reality especially for city workers, this is frequently just not possible. Therefore there exists a need and demand for flavonoid rich preparations that can be conveniently and effectively used as dietary supplements or therapeutics.

Prior art techniques for producing concentrates containing isoflavonoids from seeds generally suffer from the following drawbacks: (i) of only containing relatively low levels of isoflavones and (ii) they result in loss of raw material isoflavones and need complex multistep processing to recover them from the wastes.

The present invention seeks to overcome the shortcomings of the prior art and provide a simple and convenient method for obtaining isoflavonoids in plant concentrates at higher levels and yields compared to prior art methods.”

### DISCLOSURE OF THE INVENTION

The present invention provides a method of producing flavonoid aglycone concentrates from plant material containing a suitable flavonoid glycoside and/or conjugate thereof comprising the steps of:
(i) enzymatically converting the flavonoid glycoside or conjugate thereof into the flavonoid aglycone; and
(ii) adjusting the pH to render the flavonoid aglycone relatively insoluble and forming a concentrate containing the same.

For the purposes of the present invention the term “flavonoid” is any plant polyphenol having the general structural formula:

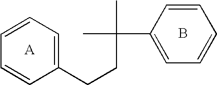

or dimers, trimers or polymers thereof.

Particular flavonoids for the purposes of the present invention include chalcones, dihydrochalones, aurones, flavanones, flavones, neoflavonoids, catechins, flavonols, dihydroflavonols, proanthocyanidins, flavans, flavan-3-ols and biflavonoids, their variously methoxylated and other modified forms such as conjugates, such as acyl conjugates and more specifically includes acacetin, apigenin, baicalein, chrysin, chrysoeriol, datiscetin, dihydrobinetin, dihydrokaempferol, diosmetin, catechin, epicatechin, eriodictyol, fisetin, fustin, galangin, hesperetin, isorhamnetin, kaempferol, luteolin/digitoflavone, morin, myricetin, naringenin, oroxylin A, ponciretin, quercetagetin, quercetin, robinetin, scutellarein, silymarin group, silybin, silidianin, silicristin, skullcapflavone II, tangeretin, wogonin, and isoflavones, such as genistein, daidzein, formononetin, biochanin A, baptenin and pratensein, having the general structural formula:

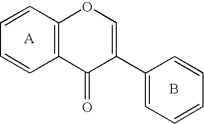

The plant material may be varied and preferably comprises a plant or part or preparation thereof that contains a flavonoid glycoside and/or a conjugate thereof. In particular, plant material includes leaves, petals, sepals, flowers, petioles, shoots, roots, stems, seeds, pods, tubers, bark, cambium, wood, galls, fruit, vegetables, herbs, bacteria, algae, ferns, sap, resins, skins such as grape, apple, onion and avocado skins, peels including citrus peels, fruit rinds, pomace such as apple, wine marc, grain hulls, straw, hay, oil seed cakes from olives, rapeseed or canola, and other oil crop extractions.

Preferably, the plant material is legume seed material such as germinating or sprouting seeds, which includes germinating seeds at the pre-sprout stage that display roots only to the stage at which sprouts are also visible. In this regard, it has been found that germinating and sprouting legume seeds can contain significant isoflavonoid levels because of (i) the initial contents of the seeds; and (ii) the isoflavones produced following germination. The significant synthesis of flavonoids does not normally commence until the germination is relatively advanced. At room temperature this is often not until after at least the second day. However the flavonoid level relative to the weight of seeds germinated plateaus after a time (usually less than ten days at room temperature) and as the seedling continue to develop to a full grown plant the actual level of isoflavones in the growing plant falls with respect to the other components such as water insoluble fibres.

Plants for the purposes of the present invention include any plant containing sufficient levels of flavonoid glycosides and/or conjugate thereof, however, particularly preferred plants are legumes such as soy (e.g. Glycine max), excluding ungerminated soya bean seeds, lupin(e)s (e.g. Lupinus spp such as L. albus, L. angutifolius L luteus, and L mutabilis, chickpeas (e.g. Cicer spp such as Cicer arietinum), pigeon peas (Canjanus cajan), white sweet clover (e.g. Meliotus alba), lucerne or alfalfa (e.g. Medicago sativa), Trifolium species. Common cooking beans (Phaseolus vulgaris and lunatus) or kitchen peas (Pisum sativum) may also be used as plant material in the present invention. Persons skilled in the art will be able to identify and obtain other raw plant material for use in the present invention without undue trial and experimentation. It will also be appreciated that a combination of material from different plants may constitute the plant material for the present invention.

Preferably, the plants used to source the plant material of the present invention produce low levels of, and even more preferably very low or no, endogenous enzymes that are able to breakdown the glycosidases or the aglycones. In this regard, many plants produce polyphenol oxidases or tyrosinases that can drastically reduce yields. Other measures may be taken to reduce the effects of unwanted enzymes in the plant material including the use of physical means such as heat or chemicals (eg sodium metabisulphite), however, the timing of these treatments must be such that the enzymes that convert the glycosidases to the aglycones are not inactivated prior to conversion of sufficient glycosidases.

The flavonoids contained within plant material are normally in the form of water soluble sugar linked glucosides and so resist concentration under conventional production of extracts such as protein concentrates. However, it is possible to use endogenous enzymes within the cells, but held in separate cellular compartments, to convert the flavonoid glycosides into aglycones.

Thus, preferably, the enzymatic conversion is achieved using endogenous enzymes contained within the plant material. When endogenous enzymes are used they may be brought into contact with the glycosides by any process that disrupts the cellular structure to allows the endogenous enzymes to come into contact with the glycoside substrates.

Thus, the present invention also provides a method of producing an enriched flavonoid concentrate from plant material containing a suitable flavonoid glycoside and/or conjugate thereof comprising the steps of:
(i) disrupting the cellular structure of the plant material to achieve enzymatic conversion of the flavonoid glycoside or conjugate thereof into the flavonoid aglycone;
(ii) adjusting the pH to render the flavonoid aglycone relatively insoluble and forming a concentrate containing the same.

Treatments to disrupt the cellular structure include treatments that rupture the cells and are varied and readily apparent to one skilled in the art. They include treatments such as grinding, crushing, pounding or rolling, freezing and thawing, enzyme treatments such as hemicellulases or cellulases, ultrasonics, drying, exposure to ultra violet light, use of pressure reduction or elevation including both extrusion and sealed batch pressure applications, microbial digestion or ensilagation, exposure to oxidising and other chemicals, detergents treatments or any combination of the foregoing.

It is to be appreciated that the texture of the raw material itself can limit the degree of cellular disruption and stronger methods would be necessary when processing material with higher fibre content or thicker cell walls, or smaller cell sizes and so on.

It will also be appreciated that any components used in the disruption process that would hinder the remainder of the process should be removed from the reaction mix prior to further processing.

It has also been found that a period of cold storage (approximately 5° C.) of sprouts reduces the level of isoflavone retained in the concentrate produced when using the same cellular disruption method, this is probably due to the cold induced changes to the cellular membranes which would make them more resistant to breaking and so limiting the mixing of the enzymes or flavonoid glycosides which are held in different portions or membrane bound organelles in the plant cells.

When the endogenous enzymes do not perform an adequate conversion of glycosides to aglycones, it may be necessary to add enzymes to improve the conversion.

Thus, the present invention also provides a method of producing an enriched flavonoid concentrate from plant material containing a suitable flavonoid glycoside and/or conjugate thereof comprising the steps of:
(i) disrupting the cellular structure of the plant material and adding additional exogenous enzyme to achieve enzymatic conversion of the flavonoid glycoside or conjugate thereof into the flavonoid aglycone;
(ii) adjusting the pH to render the flavonoid aglycone relatively insoluble and forming a concentrate containing the same.

The enzymatic conversion may be achieved with various enzymes including enzymes with the ability to hydrolyse glycoside bonds such as one or more enzyme from the group comprising glycosidases, β-glycosidases, β-galactosidase, β-glucuronidase, pectinases, hesperidinase, anthocyanase, rhamnodiastase, naringinase or takadiastase.

Other enzymes include those adapted to hydrolyse the bond in the flavonoid glycoside conjugates between the glucose (sugar) moiety and the conjugated moiety (for example an acyl group) such as the isoflavone 7-O-glycoside-6″ malonate malonylesterase or equivalent enzymes that may be found in suitable plants.

When necessary, exogenous enzymes can be obtained commercially or from sources apparent to one skilled in the art including animals such as from pig livers, plants such as Trifolium spp, Cicer spp, Helianthus spp, Melilotus spp, Medicago spp, Camellia (Thea) sinensis, Prunus spp, (eg P. amygdalus, P. communis, P. avium, P. armeniaca), Rhamnus frangula, and Rhamnus utilis, fungi such as Aspergillus spp including Aspergillus niger or Aspergillus oryzae, Saccharopolyspora erythraea, Robinia pseudoacacia L and Rhizobium spp, bacteria such as Leuconostoc oenos, Pediococcus cerevisiae and Lactobacillus plantarum or intestinal bacteria such as Bacteriodes spp and yeasts such as Saccharomyces cerevisiae, Hansenula anomala, Kloeckera apiculata and Candida pulcherimma.

Other enzymes include genetically engineered enzymes such as those obtained from genetically modified (genetically engineered) organisms. When engineered enzymes are used they be exogenous and simply added to the reaction mix. Alternatively, using genetic manipulation, material from plants which would otherwise produce insufficient amounts of endogenous enzymes or enzymes with insufficient activity could be utilized. For example, a gene encoding a suitable enzyme could be inserted into the genome of a plant, that would otherwise not produce sufficient endogenous enzyme for the conversion, and render it suitable for use in the present invention. Furthermore, genetic engineering can also be used to improve the characteristics of enzymes such as their activity. All such genetically engineered products are capable of being used in the method of the present invention.

It will be appreciated that a plurality of enzymes, either simultaneously or sequentially, may be used to effect the conversion. A plurality of enzymes may be particularly necessary when the glycoside requires conversion to an intermediate prior to conversion to an aglycone. However, a plurality of enzymes may also be used when there is no need for conversion to an intermediate. In this regard, depending on the starting material a plurality of different enzymes may achieve a better conversion than a single type of enzyme.

One of ordinary skill in the art is able to determine the nature of the enzymes required (endogenous or exogenous) for the conversion based at least on the requirements of the process and the starting material. In particular, the requirement for conversion to an intermediate and the particular enzymes used will be apparent to one skilled in the art. For example, narangin (a glycoside) must first be converted to prunin (intermediate glycoside) using alpha-rhamnosidase, and then to its flavonoid aglycone form naringinin by the hydrolysis of glucose moieties using a β glucosidase.

The amount of time required for adequate conversion to aglycones varies depending on the plant material the enzymes used, the temperature and the overall process requirements (i.e. what final levels of flavonoids in the concentrate are required). For example, in crushed Albus lupine sprouts the conversion has been found to take less than sixteen minutes at room temperature. Preferably, the conversion of the flavonoid glycoside and/or conjugate thereof is complete. However, it is more likely and practical that a portion of the flavonoid glycoside and/or conjugates thereof in the starting material will not be converted to flavonoid aglycones. Clearly, the higher the degree of conversion, the more flavonoid aglycones that will be recovered from the extraction process. In any event the level of conversion achieved in the method of the invention will be determined by the operating parameters, including the required output of the process.

When the plant material is germinating sprouts, the flavonoid levels in the germinating sprouts can be affected by the variety and quality of the seeds, germination temperature and time, as well as the presence of light and the soaking water pH. The plant material may also be specifically pretreated to increase the glycoside levels prior to exposure to the method of the present invention. For example, the plant material may be treated with copper solutions, jasmonoids, fungal extracts or sugar solutions to increase the endogenous isoflavone levels in the material. Alternatively, physical stress, such as cutting, applied to the cotyledons can also cause the plant material such as seeds to increase their production of isoflavones.

Thus, preferably, the plant material is exposed to light such as sunlight prior to further increase the endogenous isoflavone levels prior to production of the concentrates of the present invention.

The plant material may also be pretreated to remove one or more sugar residues or portions thereof from the glycoside, prior to enzymatic conversion to the flavonoid aglycone. In this regard, the flavonoid glycoside may be treated to hydrolyse some of the sugar residues, or portions thereof such as saccharide units, to yield a partially converted flavonoid glycoside. In this option, one or more sugar residues may be removed from the flavonoid glycoside by hydrolysis using strong acids that leave at least one sugar residue on the flavonoid glycoside.

Other variables may need to be adjusted to achieve the optimum performance from a given extraction process and more particularly the enzymatic conversion. The control of these variables and the particular combination of conditions that will result in the best conversion is readily apparent to one skilled in the art. Such variables include temperature, moisture content and addition of other solutes or enzyme stabilizing agents.

It will also be appreciated that extracts produced according to the method of the present invention may be treated further to further increase the concentration levels of the flavonoids of interest. In this regard, additional purification protocols may be carried out such as alcohol leaching.

Once the flavonoid aglycone has been produced it may be necessary to protect it from polymerisation or other unwanted modification. For example, polyphenol oxidase activity may need to be limited or removed to prevent polymerisation of the flavonoid aglycone. This may be achieved by physical means eg heat, or chemical means eg sulphur dioxide, sodium metabisulphite, hydrocyanic acid, carbon monoxide, protein digesting enzyme or enzymes; and/or by the use of methods to exclude oxygen, e.g. by providing an atmosphere of carbon dioxide, or nitrogen, or by vacuum suction. In the latter approach the exclusion of oxygen being maintained until the polyphenol oxidase activity can be conventionally permanently eliminated or alternatively until the flavonoid aglycone has been separated from the liquid or solids containing the polyphenol oxidase enzyme.

The pH is adjusted to render the flavonoid aglycone insoluble. Preferably, the pH is adjusted to at least about 2 pH units less than the lowest pKa value of the flavonoids to be extracted. For example, pH 5.2 or lower for genestein and biochanin A. Even more preferably, the pH is adjusted to about 4-4.5 such as 4.1 or 4.2.

The adjustment of the pH to render the flavonoid aglycone insoluble may be achieved in any one of a number of ways apparent to one skilled in the art including the addition of an acid such as hydrochloric acid, sulphuric acid, phosphoric acid, nitric acid, lactic acid, tartaric acid, citric acid, acetic acid, or propionic acid, which may be in liquid, solid or gaseous form. The pH is altered to ensure a sufficient proportion of the flavonoid aglycone is rendered insoluble. If required, the pH adjustment can be conducted with agitation to ensure thorough mixing of the reactants and the most practically complete acidification of the flavonoid aglycones possible. The soluble fraction may be treated to further to achieve a more complete retention of the flavonoid aglycone in the insoluble phase.

After the flavonoid aglycone is sufficiently present in a suspension or a precipitation the acid water soluble components can be removed and the depleted material dried to yield the flavonoid enriched concentrate. Concentration is achieved by removal of acidic water soluble components present such as sugars, minerals, saponins, amino acids and peptides.

The extraction of the acid water soluble components are rate controlled by the physical parameters of the plant material and can be carried out in a number of different ways from simple soaking and filtering, passing the acidic water solution down through the material retained on a screen using gravity or a more forced extraction approach such as counter-current extraction. Other means and methods for extracting the water soluble components will be apparent to those skilled in the art.

Drying of the leached material to form the final concentrate can be done by any one of a number of methods provided that it is fast enough to prevent microbial spoilage and temperature is not excessive to the extent that it causes undesirable flavours or reduces the food value and digestibility excessively such as by heat damaging the protein component. Spray drying is one possibility, however, other methods are apparent to those skilled in the art.

Optionally, following the conversion of the glycoside to the aglycone, the reaction mix may be stored with or without drying until further processing is convenient. In the event of the material containing levels of isoflavone destroying enzymes such as polyphenol oxidase, these would be deactivated first.

One potential complication of using plant material as the starting material is the co-precipitation of unwanted plant proteins during the concentration. In this regard, the various conditions manipulated during the method to separate the flavonoid aglycone may not adequately separate it from plant proteins. This may be addressed by additional treatment steps applied to the starting material or during the process to at least decrease the problems associated with co-precipitation.

Thus, the present invention may further comprise a treatment in which the unwanted proteins are modified so that they do not unduly dilute the concentration of the flavonoid aglycone in the method of the present invention. Such treatments include those that achieve an increased level of unwanted proteins or protein material in the soluble phase after the acidification step.

The treatments may be varied and include those readily apparent to one of ordinary skill. Treatments encompassed by the present invention include: chemical treatment eg hydrolysis, enzyme treatment of the plant material before the acid pH adjustment. Alternatively, the reaction mix following the enzymatic conversion could be passed through a column packed with a material that absorbs proteins but not flavonoid aglycones.

Preferably, the levels of the non-flavonoid proteins in the final concentrate are reduced by rendering the flavonoid aglycones insoluble at a pH particularly specific for the aglycones. Preferred pHs for this purpose are between about 1 and 3, even more preferably between about 1.5 and 2.5. It has also been found that using hydrochloric or phosphoric acid to adjust the pH is preferred to the use of sulphuric acid as these acids have been found to solubilize proteins more effectively.

As an alternative or in addition to using specific pHs the method of the present invention may also include the step of using hydrolysing enzymes to selectively or preferentially breakdown contaminating proteins prior to rendering the aglycones insoluble. This step also improves the levels of flavonoids in the final concentrate.

Thus, the reaction mix resulting from the cellular disruption step may be treated with a proteinase such as pepsin or papain that converts the unwanted proteins to forms soluble in acidic media. Size exclusion chromatography may also be used including gel filtration or a size exclusion membrane filter with pores small enough to permit flavonoid molecules but not the larger proteins through could be employed. Other biological means may also be used including fermentation with protein digesting or absorbing microbes. Ensilagation of the crushed material may also assist in the extraction protocol.

The applicant has also determined that various steps may be taken to manipulate the levels of other components in the reaction mix to maximise the concentration of flavonoids in the final concentrate.

Lipid levels in the concentrates can be reduced by physical separation from the reaction mix or by organic solvent extraction. Preferably, the solvent or solvent mixture used should be selected to minimise co-extraction of the flavonoids of interest.

Preferably, lipid levels are reduced by utilising plant biochemical behaviour. In this regard, it has been found that employing a cooling step after germination and sprouting, reduces the levels of lipids retained in the concentrate. Thus, the present invention also provides a method of producing a flavonoid aglycone concentrate from plant material in the form of germinating sprouts containing a suitable flavonoid glycoside and/or conjugate thereof comprising the steps of:
(i) cooling the germinating sprouts for a predetermined time at a predetermined temperature;
(ii) enzymatically converting the flavonoid glycoside or conjugate thereof into the flavonoid aglycone; and
(iii) adjusting the pH to render the flavonoid aglycone relatively insoluble and forming a concentrate containing the same.

The predetermined time and temperature may be varied depending on the type of plant material, the desired concentration in the final concentrate. Preferably, the temperature is at least as low as 10° C. and more preferably at least as low as 6° C. Preferably, the time is at least 1-6 weeks. However, it will be appreciated that the time and temperature for a particular extraction may be determined by a person skilled in the art using routine trial and experimentation.

With respect to carbohydrates including dietary fibres, while in some cases dietary compounds may be valued as components of a food additive for their perceived health benefits in others it may be desired to decrease these for bioavailability reasons, to raise the relative levels of flavonoids and possibly the protein also, or to enable more effective removal of water soluble components. The carbohydrate levels may be reduced by using enzyme preparations capable of breaking down the carbohydrates. Such enzyme preparations include those containing hemicellulase or cellulase.

The carbohydrate levels are preferably manipulated after or during the conversion of the glycosides to the aglycones and before the pH adjustment step. As an alternative to improving the flavonoid levels by reducing the levels of unwanted proteins in the concentrate, the nutritional appeal may be increased by increasing the total protein content in the concentrate. In this regard, depending on the desired end use of the protein concentrate, high total protein levels as well as high flavonoid levels may be desirable. To maximise total protein content consideration must be given to proteases in the reaction mix that can act to catabolise protein and therefore reduce protein yields. For example, in Albus lupine sprouts there is a protease whose pH maximum pH 4.0 approximates that of the probable pH of the protein insolubility maximum of pH 4.0 to 4.5.

Thus, the present invention may also include the step of inactivating proteases in the reaction mix. The proteases may be inactivated by heating the reaction mix that has the added advantage of increasing the precipitation of the protein and thus easing its separation from the soluble fraction. The temperature may be varied and preferably is at least 45° C. Alternatively, the proteinases may be inactivated by chemical means provided the chemicals are added after sufficient conversion of the glycosides to aglycones.

Alternatively, the effect of endogenous proteases may be limited by using plant material from cultivars with low endogenous levels of proteinases or by manipulating the growing times and/or temperature at which germination and sprouting is carried out.

The present invention also provides for the use of coagulation agents or other compounds which maximise protein insolubility such as added gums and polymeric anions eg gum arabic, carboxymethylcellulose, polygalactouric acid, alginate, carrageenans and hexametaphosphate, divalent cations such as calcium, magnesium and zinc. These agents may be added to improve the retention of protein from the reaction mix and thus can increase the amount of protein in the resulting concentrate.

Throughout the specification, unless the context requires otherwise, the word “comprise” or variations such as “comprises” or “comprising”, will be understood to imply the inclusion of a stated integer or group of integers but not the exclusion of any other integer or group of integers.

The present invention will now be described with reference to the following examples that are in no way limiting on the preceding paragraphs.

### EXAMPLES

### Example 1

### Production of Isoflavonoid Enriched Concentrates from Sprouted Albus Lupines

Bitter White Italian Lupines (Lupinus albus), provisionally identified as Tasmanian grown Superlupe cultivar, average seed weight 0.7 g, were soaked for 24 hours with two 1 hour air breaks and then soaked for approximately 1 hour every 12 hours thereafter.

The lupines were exposed to low intensity indirect sunlight and allowed to sprout at a room temperature of approximately 20 to 25° C., care being taken to separate off decaying non-viable seeds and keep a low stack height. On the twelfth day when the roots were well developed with some cotyledons opened up almost completely and primary leaves opening out, the sprouts were processed in a blender. 56 sprouts free from any attached hulls, weighing 191 grams, were divided into two batches and blended with an equal weight of water for 3 minutes.

After allowing 30 minutes from the finish of the second blending for the enzymatic hydrolysis of the isoflavone glycosides the slurry was further diluted with an additional 400 mls of water and the pH adjusted to pH 4.2. The suspension so produced was agitated from time to time during the next 22 minutes. After standing overnight the suspension was filtered on Whitman number 1 filter paper. After filtration was completed the retained material was rinsed with fresh pH 4.2 solution several times (volume used approximately 390 mls) over a period of hours.

After a day the filtered material was dried with fan forced air at 68° C., yielding 10.11 g of material with a moisture content of 3.2 g/100 g. Allowing this to come to a moisture level of 10% moisture level yields: 10.9 g of isoflavone enriched lupine concentrate with the following profile.

TABLE 1A

| Moisture | 10 | g |
|---|---|---|
| Isoflavone content | 2020 | mg |
| Genistein approximately | 1620 | mg |
| Protein (as N × 6.25) | 33.4 | g |
| Protein (as N × 5.83) | 31.2 | g |
| Lipids | 12.0 | g |
| Ash | 1.06 | g |
| Carbohydrate (dietary fibre etc) by | 43.7 | g |
| difference | | |
| [100 minus moisture, true protein, lipids, | | |
| ash/minerals, isoflavones]. | | |
| Isoflavone level in concentrate on air | 2.24 g/100 g. | |
| dry weight basis | | |
| Concentrate yield per 100 g seeds | 28 | g |
| Isoflavone yield per 100 g lupine seeds | 562 | mg |

Soaking this concentrate with light petroleum spirits (approximately 750 mls, warmed to ca 45-50° C.) for 7 hours leached out 0.98 g lipids and approximately 125 mg of isoflavones.

Drying with fan forced air at 68° C. yielded 9.01 g of material. Allowing the dried material to come to equilibrium with room moisture increased the weight to 9.67 g, with a composition per 100 g of:

TABLE 1B

| | | |
|---|---|---|
| Moisture | 10.1 | g |
| Isoflavone content | 790 | mg |
| Protein (as N × 6.25) | 37.7** | mg |
| Protein (as N × 5.83) | 35.2 | g |
| Lipids | 3.5 | g |
| Ash | 1.2 | g |
| Carbohydrate (dietary fibre etc) by | 49.1 | g |
| difference | | |
| [100 minus moisture, true protein, lipids, | | |
| ash/minerals, isoflavones]. | | |

**41.9 g/100 g dwb.

Concentrate yield per 100 g seeds-24.7 g.

### Example 2

### Production of Isoflavonoid Enriched Concentrates from Sprouted Albus Lupines, with a Heating Step to Increase Protein Retention

Bitter White Italian Lupines (Lupinus albus), provisionally identified as Tasmanian grown Superlupe cultivar were soaked for 24 hours with two 1 hour air breaks and then soaked for approximately 1 hour every 12 hours thereafter. Care was taken to separate the germinating seeds from the non-viable seeds and to ensure that the individual seeds had plenty of germination room.

The lupines were exposed to low intensity indirect sunlight and allowed to sprout at a room temperature of approximately 20 to 25° C. and on the twelfth day when the roots were well developed with some cotyledons opened up almost completely and primary leaves opening out, the sprouts were processed in a kitchen blender. 56 sprouts freed from any attached hulls weighting 182 grams, were divided into two batches and blended with an equal weight of water for four minutes.

After allowing thirty minutes from the finish of the second blending for the enzymatic hydrolysis of the isoflavone glycosides the slurry was further diluted with an additional 400 mls of water and the pH adjusted to pH 4.2. The suspension so produced was agitated from time to time during the next twenty five minutes The suspension was then heated to approximately 62.5° C. for forty minutes to at least partially coagulate the protein. After standing overnight the suspension was filtered on Whitman number 1 filter paper. After filtration was completed the retained material was rinsed with fresh pH 4.2 solution several times (volume used approximately 640 mls) over a period of hours.

After a day the filtered material was dried with fan forced air at 68° C., yielding 14.4 g of material with a moisture content of 3.1 g/100 g. Allowing this to come to a moisture level of 10% moisture level yields 15.5 g of isoflavone enriched lupine concentrate of:

TABLE 2A

| Moisture | 10 | g |
|---|---|---|
| Isoflavone content | 1340 mg (1.34 g/100 g) | |
| Genistein approximately | 1080 | mg |
| Protein (as N × 6.25) | 29.6* | g |
| Protein (as N × 5.83) | 27.6 | g |
| Lipids | 18.3 | g |
| Ash | 0.90 | g |
| Carbohydrate (dietary fibre etc) by | 41.9 | g |
| difference | | |
| [100 minus moisture, true protein, lipids, | | |
| ash/minerals, isoflavones]. | | |
| Isoflavone level in concentrate on air | 1.44 g/100 g. | |
| dry weight basis | | |
| Concentrate yield per 100 g seeds | 39.5 | g |
| Isoflavone yield per 100 g lupine seeds | 529 | mg |

*protein dry weight basis 32.9 g/100 g

Soaking the concentrate with petroleum spirits (approximately 750 mls, warmed to approximately 45-50° C.) for seven hours leached out 2.58 g lipids and approximately 127 mg isoflavones.

Drying with fan forced air at 68° C. yielded 11.69 g of material. Allowing the dried material to come to equilibrium with room moisture increased the weight to 12.69 g, with a composition per 100 g of:

TABLE 2B

| | | |
|---|---|---|
| Moisture | 10.5 | g |
| Isoflavone content | 457 | mg |
| Protein (as N × 6.25) | 36.2** | mg |
| Protein (as N × 5.83) | 33.8 | g |
| Lipids | 2 | g |
| Ash | 1.1 | g |
| Carbohydrate (dietary fibre etc) by | 52.1 | g |
| difference | | |
| [100 minus moisture, true protein, lipids, | | |
| ash/minerals, isoflavones]. | | |

**40.4 g/100 g dwb.

Concentrate yield per 100 g seeds-32.4 g.

Assuming that the albus lupines had the Australian average protein contents of 39.5 g/100 g seeds dwb, though the level can be as low as 31.8 g/100 g dwb, then

The heating (deactivation of acid pH protease?) in raising the protein retention efficiency from 9.3 g to 13.1 g per 100 g dried seeds or approximately a third or higher of the expected original lupine seed protein. Increasing the efficiency of protein retention protein by reduction of the time before enzyme deactivation, use of complexing cations can be expected to rise to the efficiency seen for commercial legume protein concentrate production of retaining approximately one half of the original protein.

### Example 3

### Production of Isoflavonoid Enriched Concentrates from Sprouted Albus Lupines, after Storage at a Low Temperature, with a Heating Step to Increase Protein Retention

Bitter White Italian Lupines (Lupinus albus), provisionally identified as Tasmanian grown Superlupe cultivar were soaked for 24 hours with two 1 hour air breaks and then soaked for approximately 1 hour every 12 hours thereafter. Care was taken to separate the germinating seeds from the non-viable seeds and to ensure that the individual seeds had plenty of germination room.

The lupines were exposed to low intensity indirect sunlight and allowed to sprout at a room temperature of approximately 20 to 25° C. and on the twelfth day when the roots were well developed with some cotyledons opened up almost completely and primary leaves opening out, the sprouts were placed in moist soaked paper lined containers and stored at 6° C. for eight days.

After the temperature was allowed to adjust to room temperature (25.0° C.) the sprouts were removed from the containers and processed in a kitchen blender (Panasonic model Super Blender). 63 sprouts freed from any attached hulls and weighted on average 3.41 grams per sprout, were divided into two batches and blended with an equal weight of water for three minutes on the liquefy option.

After allowing 33 minutes from the finish of the second blending for the enzymatic hydrolysis of the isoflavone glycosides the slurry was further diluted with an additional 400 mls of water and the pH adjusted to pH 4.1. The suspension so produced was agitated from time to time during the next twenty minutes. The suspension was then heated to approximately 62.5° C. for 45 minutes to at least partially coagulation the protein. After standing overnight the suspension was filtered on Whitman number 1 filter paper. After filtration was completed the retained material was rinsed with fresh pH 4.2 solution several times (volume used approximately 680 mls) over a period of hours.

After a day the filtered material was dried with fan forced air at 68° C., yielding 9.86 g of material with a moisture content of 2.5 g/100 g. Allowing this to come to a moisture level of 10% moisture level yields 10.67 g of isoflavone enriched lupine concentrate of:

TABLE 3A

| Moisture | 10 | g |
|---|---|---|
| Isoflavone content | 1890 mg (1.9 g/100 g) | |
| Genistein approximately | 1520 | mg |
| Protein (as N × 6.25) | 26.2* | g |
| Protein (as N × 5.83) | 24.4 | g |
| Lipids | 7.56 | g |
| Ash | 1.2 | g |
| Carbohydrate (dietary fibre etc) by | 55.5 | g |
| difference | | |
| [100 minus moisture, true protein, lipids, | | |
| ash/minerals, isoflavones]. | | |
| Isoflavone level in concentrate on air | 2.1 g/100 g. | |
| dry weight basis | | |
| Concentrate yield per 100 g seeds | 24.2 | g |
| Isoflavone yield per 100 g lupine seeds | 457 | mg |

*protein dry weight basis 29.1 g/100 g

Soaking the concentrate with petroleum spirits (approximately 750 mls, warmed to approximately 45-50° C.) for seven hours leached out 0.46 g lipids and approximately 98 mg isoflavones.

Drying with fan forced air at 68° C. yielded 9.30 g of material. Allowing the dried material to come to equilibrium with room moisture increased the weight to 10.00 g, with a composition per 100 g of:

As product allowed to come to equilibrium with room moisture, per 100 g:

TABLE 3B

| | | |
|---|---|---|
| Moisture | 9.5 | g |
| Isoflavone content | 896 | mg |
| Protein (as N × 6.25) | 27.9** | mg |
| Protein (as N × 5.83) | 26.1 | g |
| Lipids | 2.8 | g |
| Ash | 1.3 | g |
| Carbohydrate (dietary fibre etc) by | 59.2 | |
| difference | | |
| [100 minus moisture, true protein, lipids, | | |
| ash/minerals, isoflavones]. | | |

**30.8 g/100 g dwb.

Concentrate yield per 100 g seeds-22.7 g.

Analysis of the acid insoluble isoflavone aglycones extracted from crushed sprouted albus lupines, dried and extracted with methanol, analysed by proton nuclear magnetic resonance and high pressure liquid chromatography combined with ultra-violet spectroscopy, shows the majority to be genistein with a smaller amount of 2′-hydroxygenistein, and small amounts of other isoflavonoids.”

The method of the present invention allows for the production of significant amounts of the specific isoflavone aglycones using a relatively simple process that is amenable to scale up for the large scale production of flavonoid concentrates for use as feed and or dietary supplements. Concentrates containing isoflavones are made conventionally from defatted soya material and are found to contain the three isoflavones in the order genisteins greater than daidzeins greater than glycitrins. When a range of sprouted legumes are cellular disrupted they are found to not only yield significantly higher levels of acidic solution insoluble aglycone isoflavones per amount of original seed, up to over six fold greater than the total isoflavone content of soybean seeds, but the isoflavone species make-up can also differ drastically.

Thus, the present invention offers the potential for concentrates with higher isoflavone contents but different make-ups such as with greater amount of daidzein than genistein (sprouted soybeans), or with the genistein isoflavone proportion much higher (both albus and angustifolius lupine cultivar sprouts), modified genisteins (both albus and angustifolius lupine cultivar sprouts) and concentrates in which the isoflavones are essentially one to one or two to one formonentin to biochanin A (desi and Kabuli chickpea sprouts respectively).

The isoflavones are not biochemically identical and differ in their effects and health benefits and so the demand will not be limited to a single isoflavone combination make-up. The approach of utilising germinated legume sprouts enables the generation of more market segment tailored products.

### Example 4

### Production of Isoflavonoid Enriched Concentrates from Sprouted Angustifolius Lupines

Angustifolius (Narrow leaf) lupines (Lupinus angustifolius), of the gungurru cultivar about six months post harvest were obtained from a commercial grain exporter (average seed weight 0.15 g) were soaked for 24 hours with two 1 hour air breaks and then soaked for approximately 1 hour every 12 hours thereafter.

The lupines were allowed to sprout at a room temperature of approximately 25° C., and exposed to low intensity indirect sunlight. After eight days, whole sprouts were separated from the ungerminated seeds, and placed in moist soaked paper lined containers and stored at 6° C. for five and a half days.

The lupines were allowed to sprout at a room temperature of approximately 25° C. At this stage the lupine cotyledons halves had opened up and were wide apart, and some of the primary leaves had developed to the point of separating leaflets but not the point that the leaflets had flattened out. Stems approximately 6 to 7 cm long, roots length taken from top of colour change up to 8.8 cm long.

After the temperature was allowed to adjust to room temperature (25° C.) the sprouts were removed from the containers and processed in a kitchen blender (Panasonic model Super Blender). 468 sprouts, freed from any attached hulls and weighing on average 1.14 grams (per sprout), were divided into four batches and blended with an equal weight of water for over three minutes on the liquefy option. The first and second blendings and the third and fourth were combined to yield two batches of approximately 534 g each. At this point the temperature of the suspensions were 32° C. and 33° C., respectively.

After allowing 90 minutes from the finish of the blending for the enzymatic hydrolysis of the isoflavone glycosides the two final suspensions (slurries) were further diluted in additional water to a final weight of 800 g and the pH was adjusted to pH 4.5. The suspension so produced was agitated from time to time during the next sixty minutes and then the suspensions were filtered on coarse paper. After filtration was completed the retained material was rinsed with fresh pH 4.5 solution several times (volume used approximately 500 ml) over a period of two and a half hours.

After a day the filtered material was dried with fan forced air at 68° C., then allowed to come to equilibrium with the air moisture, yielding 24.57 g and 22.62 g material respectively, equivalent to 70 g and 64.4 g per 10 g of the original seeds. Hexane extractable lipid content of concentrate measured approximately 5.3 g/100 g.

The level of isoflavones in the air dry material was 268 mg/100 g, and 305 mg/100 g respectively, or the equivalent of 188 mg and 196 mg per 100 g of original seeds.

Analysis of the acid insoluble isoflavone aglycones extracted from crushed sprouted gungurru angustifolius lupines, dried and extracted with methanol, analysed by proton nuclear magnetic resonance and high pressure liquid chromatography combined with ultra-violet spectroscopy, showed the majority (about two thirds) to be genistein with a smaller amount of 2′-hydroxygenistein, and small amounts of formononetin, and singly and doubly prenylated isoflavonoids.

### Example 5A

### Production of Isoflavonoid Enriched Concentrates from Fresh (Non Cool Stored) Sprouted Soya Beans

Soya beans (Glycine max) of unknown cultivar were purchased from a bulk food ingredients shop, the seeds were not size graded (average seed weight 0.175 g) were soaked for 24 hours with two 1 hour air breaks and then soaked for approximately 1 hour every 12 hours thereafter.

The soya beans were allowed to sprout at a room temperature of approximately 25° C., and exposed to low intensity indirect sunlight. After six and a half days most of the sprouts had forced off their seed coats and the cotyledons were green and bending towards the horizontal on vertical stems, some cotyledons opening but no emergence of primary leaves. Roots up to 6 cm long, stems up to 8 cm long.

The sprouts were processed in a kitchen blender (Panasonic model Super Blender). 269 sprouts free from any attached hulls, weighing 189.5 grams, were blended with an equal weight of water for over 3 minutes. At the end of the blending the temperature was 35.4° C.

After allowing an hour from the finish of the second blending for the enzymatic hydrolysis of the isoflavone glycosides the slurry was further diluted with an additional 400 ml of water and the pH adjusted to pH 4.5. After standing overnight at 5° C. the suspension was filtered on coarse paper. After filtration was completed the retained material was rinsed with fresh pH 4.5 solution three times (volume used approximately 400 ml) over a period of two hours.

After a day the filtered material was dried with fan forced air at 68° C., then allowed to come to equilibrium with the air moisture, yielding 24.57 g material equivalent to 54.7 g per 100 g of the original seeds. Hexane extractable lipid content of concentrate measured approximately 21.6 g/100 g.

The level of isoflavones in the air dry material was 680 mg/100 g, or the equivalent of 370 mg per 100 g of original seeds.

### Example 5B

### Production of Isoflavonoid Enriched Concentrates from Cool Stored Sprouted Soya Beans

The same plant material as in example 5A, that is soya beans (Glycine max) of unknown cultivar was purchased from a bulk food ingredients shop, the seeds were not size graded, average seed weight 0.175 g, were soaked for 24 hours with two 1 hour air breaks and then soaked for approximately 1 hour every 12 hours thereafter.

The soya beans were allowed to sprout at a room temperature of approximately 25° C., and exposed to low intensity indirect sunlight. After six and a half days sprouts were separated from ungerminated seeds and placed in moist soaked paper lined containers and stored at 6° C. for six and a half days.

After the temperature was allowed to adjust to room temperature (25° C.) the sprouts were removed from the containers and processed in a kitchen blender (Panasonic model Super Blender). At this stage the majority of the cotyledons were just starting to separate but remained pressed together at the outer ends, a few had primary leaves rising from between the cotyledon halves. Stems were up to 11 cm long and the roots up to 8 cm long.

298 sprouts free from any attached hulls, weighing 253.7 grams, were blended with an equal weight of water for over 3 minutes. At the end of the blending the temperature was 35° C.

After allowing an hour and a quarter from the finish of the second blending for the enzymatic hydrolysis of the isoflavone glycosides the slurry was further diluted with an additional 400 ml of water and the pH adjusted to pH 4.5. After standing for an hour the suspension was filtered on coarse paper. After filtration was completed the retained material was rinsed with fresh pH 4.5 solution once (volume used approximately 200 ml) over a period of two hours.

After a day the filtered material was dried with fan forced air at 68° C., then allowed to come to equilibrium with the air moisture, yielding 24.64 g material equivalent to 47.3 g per 100 g of the original seeds. Hexane extractable lipid content of concentrate measured approximately 13.3 g/100 g.

The level of isoflavones in the air dry material was 450 mg/100 g, or the equivalent of 236 mg per 100 g of original seeds.

Analysis of the acid insoluble isoflavone aglycones extracted from crushed sprouted soya beans, dried and extracted with methanol, analysed by proton nuclear magnetic resonance and high pressure liquid chromatography combined with ultra-violet spectroscopy, showed the isoflavonoids to be a minority of genistein (about 28%) and the rest, about 72%, daidzein with formononetin.

### Example 6

### Production of Isoflavonoid Enriched Concentrates from Sprouted Kabuli Chickpeas

Kabuli or gabanzo class Chickpeas (Cicer arietinum) of unknown cultivar (average weight 0.51 g) were purchased from a Mediterranean food ingredients shop, were soaked for 24 hours with two 1 hour air breaks and then soaked for approximately 1 hour every 12 hours thereafter.

The chickpeas were allowed to sprout at a room temperature of approximately 25° C., and exposed to low intensity indirect sunlight. After ten and a half days the sprouts were at the stage of between 3 and 4 leaflets on the stems. The cotyledons had not all opened, the seed coats being too restrictive. Shoots up to 4.5 cm long, roots up to 8 cm long, with well developed side roots up to 1.7 cm long.

The sprouts were processed in a kitchen blender (Panasonic model Super Blender). 99 dehulled sprouts weighting 130 g were blended with an equal weight of water for 3 minutes.

After allowing an hour from the finish of the blending for the enzymatic hydrolysis of the isoflavone glycosides the slurry was further diluted with an additional 260 ml of water and the pH adjusted to pH 4.5. The slurry was allowed to sit for about two hours at room temperature and then stored at 0° C. for a further hour and a half before being filtered on coarse paper followed by rinsing the retained solids with batches of pH 4.5 solution water, total volume of approximately 500 ml.

After a day the filtered material was dried with fan forced air at 65° C., then allowed to come to equilibrium with the air moisture, yielding 36.81 g material equivalent to 72 g per 10 g of the original seeds. Hexane extractable lipid content of concentrate measured approximately 9.9%.

The level of isoflavones in the air dry material was 915 mg/100 g, or the equivalent of 660 mg per 100 g of original seeds.

Analysis of the acid insoluble isoflavone aglycones extracted from crushed sprouted kabuli chickpeas, dried and extracted with methanol, analysed by proton nuclear magnetic resonance and high pressure liquid chromatography combined with ultra-violet spectroscopy, showed the isoflavonoids to be essentially formononetin and biochanin A in a ratio of approximately 65:35 with traces of pratensein and genistein.

### Example 7

### Production of Isoflavonoid Enriched Concentrates from Sprouted Desi Chickpeas

Desi class Chickpeas (Cicer arietinum) of unknown cultivar, ungraded for size but with an average weight of 0.121 g, were purchased from a Mediterranean food ingredients shop, soaked for 24 hours with two 1 hour air breaks and then soaked for approximately 1 hour every 12 hours thereafter.

The chickpeas were allowed to sprout at a room temperature of approximately 25° C., and exposed to low intensity indirect sunlight. After seven and a half days the sprouts were at the third leaf bracket stage at the top of the stem, roots and sprouts were of variable length but roots were up to 7.2 cm long and stems up to 4.1 cm long.

The sprouts were processed in a kitchen blender (Panasonic model Super Blender). 410 dehulled sprouts weighting 183 g were blended with an equal weight of water for 3 minutes.

After allowing an hour from the finish of the blending for the enzymatic hydrolysis of the isoflavone glycosides the slurry was further diluted with an additional 376 ml of water and the pH adjusted to pH 4.5. After another two and a quarter hours the suspension was a filtered on coarse paper followed by rinsing the retained solids with batches of pH 4.5 solution water, total rinsing volume was approximately 350 ml.

After a day the filtered material was dried with fan forced air at 68° C., then allowed to come to equilibrium with the air moisture, and then the material yielding 33.67 g material equivalent to 68 g per 100 g of the original seeds. Hexane extractable lipid content of concentrate measured approximately 6.4 g/100 g.

The level of isoflavones in the air dry material was 428 mg/100 g, or the equivalent of 290 mg per 100 g of original seeds.

Analysis of the acid insoluble isoflavone aglycones extracted from crushed sprouted desi chickpeas, dried and extracted with methanol, analysed by proton nuclear magnetic resonance and high pressure liquid chromatography combined with ultra-violet spectroscopy, showed the isoflavonoids to be essentially formononetin and biochanin A in a ratio of approximately 55:45 and a trace of formononetin.

Other modifications and adaptations apparent to one skilled in the art are to be encompassed within the scope of the present invention.

Throughout the specification, unless the context requires otherwise, the word “comprise” or variations such as “comprises” or “comprising”, will be understood to imply the inclusion of a stated integer or group of integers but not the exclusion of any other integer or group of integers.

The claims defining the invention are as follows:

1. A method of producing a flavonoid aglycone concentrate from plant material containing a suitable flavonoid glycoside and/or conjugate thereof comprising the steps of:
(i) enzymatically converting the flavonoid glycoside or conjugate thereof into the flavonoid aglycone, and
(ii) adjusting the pH to render the flavonoid aglycone relatively insoluble and forming a concentrate containing the same.

2. A method of producing an enriched flavonoid concentrate from plant material containing a suitable flavonoid glycoside and/or conjugate thereof comprising the steps of:
(i) disrupting the cellular structure of the plant material to achieve enzymatic conversion of the flavonoid glycoside or conjugate thereof into the flavonoid aglycone;
(ii) adjusting the pH to render the flavonoid aglycone relatively insoluble and forming a concentrate containing the same.

3. A method of producing an enriched flavonoid concentrate from plant material containing a suitable flavonoid glycoside and/or conjugate thereof comprising the steps of:
(i) disrupting the cellular structure of the plant material and adding additional exogenous enzyme to achieve enzymatic conversion of the flavonoid glycoside or conjugate thereof into the flavonoid aglycone;
(ii) adjusting the pH to render the flavonoid aglycone relatively insoluble and forming a concentrate containing the same.

4. A method of producing a flavonoid aglycone concentrate from plant material in the form of germinating sprouts containing a suitable flavonoid glycoside and/or conjugate thereof comprising the steps of:
(i) cooling the germinating sprouts for a predetermined time at a predetermined temperature;
(ii) enzymatically converting the flavonoid glycoside or conjugate thereof into the flavonoid aglycone; and
(iii) adjusting the pH to render the flavonoid aglycone relatively insoluble and forming a concentrate containing the same.
